(19) United States
(12) Patent Application Publication
(10) Pub. No.: US 2013/0261701 A1
(43) Pub. Date: Oct. 3, 2013

(54) IMPLANTABLE ACTUATOR FOR HEARING STIMULATIOIN

(71) Applicants: Thomas Kuratle, Nyon (CH); Daniel Yanisse, Poisy (FR)

(72) Inventors: Thomas Kuratle, Nyon (CH); Daniel Yanisse, Poisy (FR)

(73) Assignee: ADVANCED BIONICS AG, Zug (CH)

(21) Appl. No.: 13/882,381

(22) PCT Filed: Oct. 27, 2010
(86) PCT No.: PCT/EP10/66281

§ 371 Date: Jun. 5, 2013

(51) Int. Cl. A61F 11/04

(52) U.S. Cl. CPC A61F 11/04
USPC 607/57; 607/137

(57) ABSTRACT

There is provided an implantable actuator for stimulating a patient's hearing, comprising a support frame (70), a dielectric electroactive polymer membrane (74) fixed and tensioned on the support frame, with the polymer membrane being provided with a first electrode (76) on one side and a second electrode (78) on the other side, means (80, 82, 84) for supplying electric signals to the electrodes, wherein the polymer membrane is for vibrating the cochlear liquid according to the electric signals supplied to the electrodes by direct contact or by contact with the round window membrane (88).

### BACKGROUND OF THE INVENTION

1. Field of the Invention

The invention relates to an implantable actuator for stimulating a patient's hearing.

2. Description of Related Art

Middle ear implants (MEI) are offered for patients who cannot be treated with electro-acoustic hearing aids. Such patients usually suffer from radical middle ear cavities, atresia, otosclerosis in combination with sensorineural hearing loss, chronic infections or allergies of the ear canal. MEIs improve or bypass the middle ear ossicles by directly mechanically stimulating the cochlea. In practice, there are three types of actuators currently used: piezoelectric, electromagnetic and electromechanical actuators. Piezoelectric actuators use the properties of piezoelectric materials or crystals, i.e., when a voltage is applied, a deformation or bending of the material occurs which will provide the mechanical energy for the stimulation. Electromagnetic actuators generate a fluctuating magnetic field by a coil according to acoustic input audio signals, thereby causing a magnet attached to the ossicular chain, the tympanic membrane or the inner ear to vibrate. Electromechanical actuators are a variation of electromagnetic actuators, wherein the energizing coil and the magnet are housed within an actuator assembly which is attached to the ossicular chain, thereby optimizing the spacial and geometric relationship to avoid variability.

For such type of actuators, the major issue during surgery is the fixation of the actuator on the ossicles or the round window, since the actuator has to be precisely fixed and coupled to the coupling site in order to avoid any loss of vibration energy which would reduce hearing improvement achieved by the actuator. Also, for some types of actuators, the axis of movement of the actuator is not aligned with the direction of natural movement of the auditory component to which it is coupled. Other issues are the size of the available actuators, which makes it difficult to directly fix them on the oval or round window, and the weight of the actuator, since the loading effect has an impact on ossicle vibration. Also, a good fixation point is difficult to be found in the middle ear region.

It is known that electro-active polymers (EAPs), which are polymers exhibiting a change in size or shape when stimulated by an electric field, may be used as actuators, for example, in pumps or as a so-called artificial muscle. The article “Performance characterization of miniaturized dielectric elastomer actuators fabricated using metal ion implantation” by S. Rosset et al., IEEE 21^{st} conference on MEMS, 2008, relates to an actuator comprising a dielectric elastomer membrane made of polydimethylsiloxane (PDMS) provided on both sides with a soft electrode formed by ion implantation of the respective surface layer of the membrane, with the membrane being fixed to and stretched over a support frame formed by a silicon chip, wherein a voltage applied to the two electrodes causes the membrane to buckle. It is mentioned that such actuators may be used in micro-pumps and active optical devices.

International Patent Application Publication WO 2005/027161 A2 and corresponding U.S. Pat. No. 7,411,331 relate to an EAP actuator which may be used for motors or loudspeakers.

International Patent Application Publication WO 2007/054589 A2 and corresponding U.S. Patent Application Publication 2010/014696 relate to an EAP actuator included in a hearing protection device for adjusting the cross-section of a sound channel of the hearing protection device in order to adjust the sound attenuation provided by the hearing protection device.

International Patent Application Publication WO 2010/077465 A1 and U.S. Patent Application Publication 2006/0238066 A1 relate to the use of EAPs for loudspeakers.

International Patent Application Publication WO 2009/020648 A1 and corresponding U.S. Patent Application Publication 2010/211186 relate to the use of implanted EAP actuators as an artificial muscle for patients suffering from a paralysis or paresis.

U.S. Patent Application Publication 2006/0047180 A1 relates to the use of EAP actuators for opening or closing a body cavity.

U.S. Patent Application Publication 2009/0173352 A1 relates to an airway implant device comprising an EAP element.

### SUMMARY OF THE INVENTION

It is an object of the invention to provide for an implantable hearing stimulation actuator which can be implanted relatively easily while nevertheless providing for high performance. It is also an object of the invention to provide for a corresponding hearing stimulation method.

According to the invention, these objects are achieved by an implantable actuator and method as described herein.

The invention is beneficial in that, by providing the actuator with a dielectric electro-active polymer membrane fixed and tensioned by a support frame and being provided with a first electrode on one side and a second electrode on the other side, with the membrane directly contacting the cochlear liquid or contacting the round window membrane for vibrating the cochlear liquid, a particularly small actuator is provided which can be easily placed on the round window or which can be inserted into the cochlea for floating within the cochlear liquid, whereby actuator fixation is made easier, thereby rendering surgery less complex and enhancing stimulation quality due to reliable fixation/placement of the actuator.

These and further objects, features and advantages of the present invention will become apparent from the following description when taken in connection with the accompanying drawings which, for purposes of illustration only, show several embodiments in accordance with the present invention.

### BRIEF DESCRIPTION OF THE DRAWINGS

FIG. 1 is a sectional view of an example of a hearing instrument comprising an actuator according to the invention after implantation;

FIG. 2 is a block diagram of the hearing instrument of FIG. 1;

FIG. 3 is a sectional view of an example of an actuator according to the invention when mounted at the round window;

FIG. 4 is a schematic sectional view of an example of an actuator according to the invention illustrating the working principle of the actuator;

FIG. 5 is a sectional view of an example of an actuator according to the invention prior to implantation;

FIGS. 6a and 6b are schematic perspective views of an example of an actuator according to the invention illustrating the working principle of the actuator, without and with a voltage being applied to the actuator, respectively; and

FIG. 7 is a schematic view of an example of an actuator according to the invention which is integrated within a cochlear implant electrode arrangement.

### DETAILED DESCRIPTION OF THE INVENTION

FIG. 1 shows a cross-sectional view of the mastoid region, the middle ear and the inner ear of a patient after implantation of an example of a hearing aid according to the invention, wherein the hearing aid is shown only schematically. The system comprises an external unit 10, which is worn outside the patient's body at the patient's head and an implantable unit 12 which is implanted under the patient's skin 14, usually in an artificial cavity created in the user's mastoid 16. The implantable unit 12 is connected via a cable assembly 18 to an actuator 20 acting on the cochlea 24. The external unit 10 is fixed at the patient's skin 14 in a position opposite to the implantable unit 12, for example, by magnetic forces created by cooperating fixation magnets provided in the external unit 10 and the implantable unit 12, respectively (these magnets are not shown in FIG. 1).

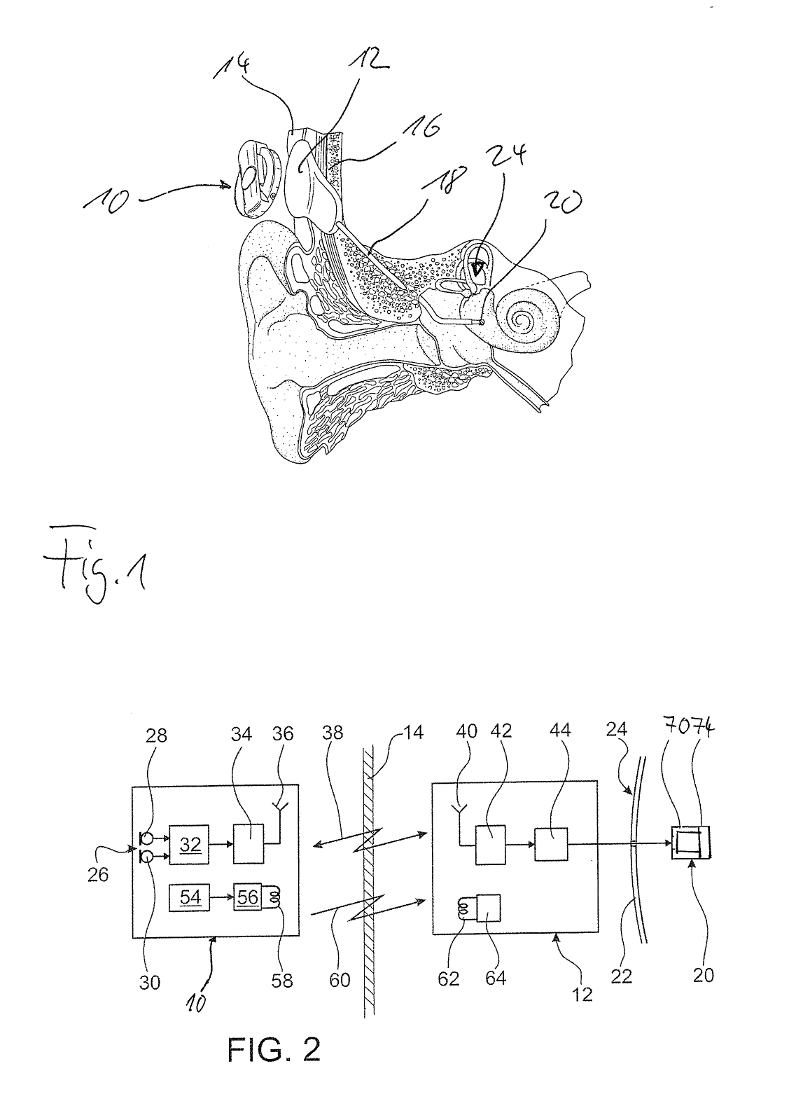

An example of a block diagram of the system of FIG. 1 is shown in FIG. 2. The external unit 10 includes a microphone arrangement 26 comprising, for example, at least two spaced-apart microphones 28 and 30 for capturing audio signals from ambient sound, which audio signals are supplied to an audio signal processing unit 32, wherein they may undergo, for example, acoustic beam-forming. The audio signals processed by the audio signal processing unit 32 are supplied to the transmission unit 34 connected to a transmission antenna 36 in order to enable transcutaneous transmission of the processed audio signals via an inductive link 38 to the implantable unit 12 which comprises a receiver antenna 40 connected to a receiver unit 42 for receiving the transmitted audio signals. The received audio signals are supplied to a driver unit 44 which drives the actuator 20.

The external unit 10 comprises a power supply 54, which may be a replaceable or rechargeable battery, a power transmission unit 56 and a power transmission antenna 58 for transmitting power to the implantable unit 12 via a wireless power link 60, The implantable unit 12 comprises a power receiving antenna 62 and a power receiving unit 64 for powering the implanted electronic components with power received via the power link 60. Preferably, the audio signal antennas 36, 40 are separated from the power antennas 58, 62 in order to optimize both the audio signal link 38 and the power link 60. However, if a particularly simple design is desired, the antennas 36 and 58 and the antennas 40 and 62 could be physically formed by a single antenna, respectively.

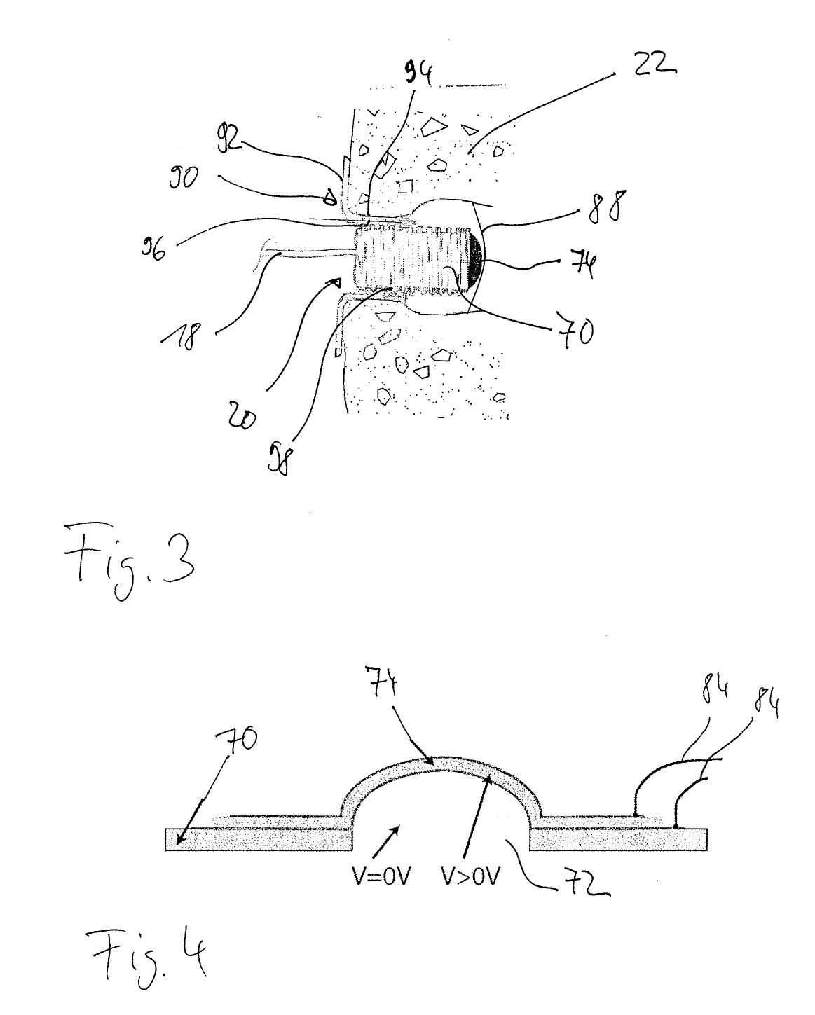

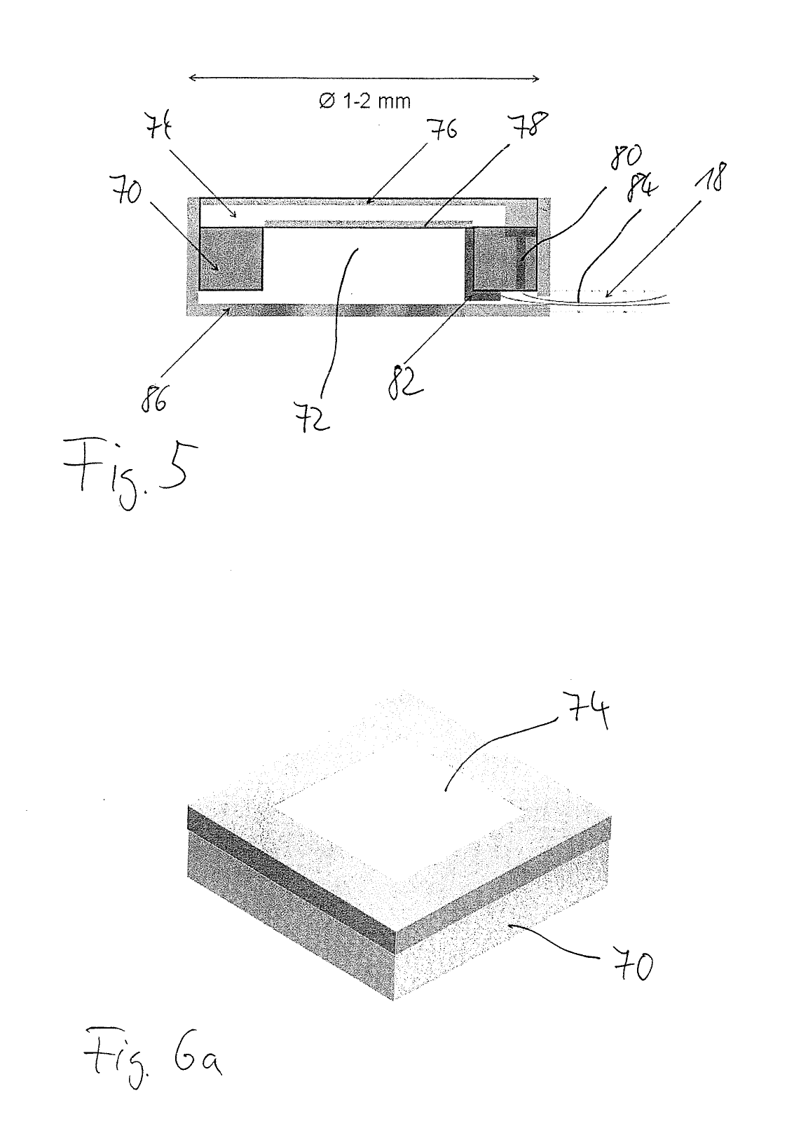

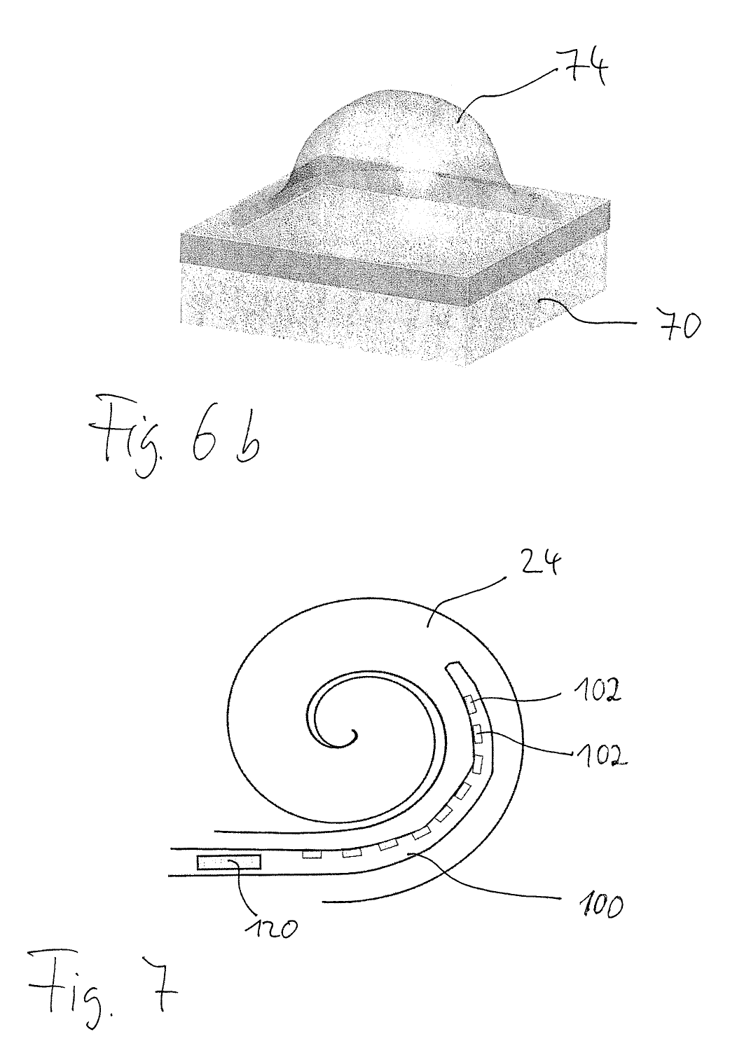

While FIG. 2 shows an example of the actuator 20 which is floating within the cochlear liquid, FIG. 3 shows an example of the actuator 20 which is fixed at the round window. FIG. 5 shows an example of a structure of the actuator 20 which is suitable both for an actuator of the type of FIG. 2 and for an actuator of the type of FIG. 3; the working principle of such actuator is illustrated in FIGS. 4 and 6.

According to FIG. 5, the actuator 20 comprises a support frame 70 formed by a micromachined silicon chip provided with an opening 72 which is covered on one side by a membrane 74 made of a dielectric electroactive polymer, for example, PDMS. The peripheral part/circumference of the membrane 74 is fixed on (by bonding) and supported by the support frame 70 in a stretched/tensioned condition. Rather than silicon, also another standard substrate, such as a glass substrate, may be used for forming the support frame 70.

The membrane 74 is provided with a first electrode 76 on one side and with a second electrode 78 on the other side. Preferably, the electrodes 76, 78 are soft electrodes which are formed by an ion implanted surface layer of the polymer membrane 74. The conductive implanted ions, for example, may be Ti ions (in the article “Performance characterization of miniaturized dielectric elastomer actuators fabricated using metal ion implantation” by S. Rosset et al., IEEE 21^{st} conference on MEMS, 2008, pages 503 to 506, details are given as to how a PDMS membrane on a silicon chip may be provided with ion implantation electrodes). The electrodes 76, 78 are connected via electrical connections 80, 82, which are patterned on the support frame 70, to wires 84 of the cable assembly 18, whereby the electrodes 76, 78 are connected to the driver unit 44 of the implantable unit 12. Thus, the audio signals provided by the driver unit 44 can be supplied to the electrodes 76, 78. By providing the electrodes 76, 78 as soft electrodes by ion implantation, mechanical properties of the membrane 74 can be essentially preserved, since ion implantation typically has less impact on mechanical properties than surface deposition or sputtering of electrodes. Thus, by preserving mechanical properties of the membrane 74, a lower voltage is needed for the same displacement of the membrane 74, compared to a membrane provided with surface deposition electrodes or sputter electrodes. Moreover, since the conductive ions are located under the surface of the membrane material, they are expected to not have a negative impact on the biocompatibility of the membrane material.

The support frame 70, the electrical connections 80, 82 and the wires 84, where necessary, are covered with a biocompatible cover layer 86 which may be made of, for example, polyetheretherketone (PEEK), titanium, silicone or PMMA. The cover layer 86 serves to isolate the support frame 70 and the electrical connections 80, 82 from the environment. PEEK is a material which already has been used to replace traditional materials such as titanium and ceramics and which can be processed by conventional methods such as injection molding and extrusion; and it can be machined in a manner allowing broad design and manufacturing flexibility. It is sterilization resistant, radiolucent and compatible with imaging techniques such as X-ray and MRI. Preferably, the wires 84 of the cable assembly 18 are protected by a silicone sheath.

Typically, the actuator 20 has as diameter of about 1 to 2 mm.

The working principle of the actuator 20 is illustrated in FIGS. 4 and 6a (without voltage applied) and 6b (with voltage applied) . As long as no voltage is applied to the electrodes 76, 78, the membrane 74 remains suspended and stretched by the support frame 70 in an essentially flat configuration (in order to ensure that the membrane 74 will buckle in the right direction, a mechanical tension may be applied, or the initial configuration may be slightly curved). If a voltage was applied to an EAP layer with free boundary conditions, it would stretch. In case of membranes clamped at their circumference, as in the case of the actuator 20, wherein such clamping is provided by the fixation of the membrane 74 on the support frame 70, area expansion is impossible, so that applying a voltage to the electrodes 76, 78 in this case will lead to compressive stress built up in the membrane 74, up to the limit of stability, at which point the membrane 74 will buckle (see FIG. 4 and FIG. 6b).

Hence, operation of the actuator 20 is based on the compression of a dielectric elastomer membrane by the electrostatic pressure due to a voltage applied to compliant soft electrodes which results in an elongation of the elastomer without a change in volume. By attaching the membrane on a stiff substrate/support frame, the elongation is transformed into buckling of the membrane.

According to the invention, such buckling of the membrane 74 is used to vibrate the cochlear liquid according to the audio signals supplied to the electrodes 76, 78, either by direct contact or by contact with the round window membrane 88, in order to stimulate the patient's hearing.

In FIG. 3, an example of an actuator 20 is shown, wherein the membrane 74 is used for vibrating the round window membrane 88. To this end, the actuator 20, i.e., more precisely, the support frame 70 thereof, is fixed at the round window in such a manner that the membrane 74 touches the round window membrane 88. In the example shown in FIG. 3, such fixation is provided by an auxiliary fixation element 90 which is fixed within the round window opening in the cochlea wall 22. The fixation element 90 comprises an outer flange portion 92 for abutting the cochlea wall 22 and an inner cylindrical portion 94 extending into the round window opening and comprising an internal thread 96 which engages with an outer thread 98 provided at the periphery of the support frame 70 in such a manner that the support frame 70 can be screwed into the cylindrical portion 94 of the fixation element 90. Thereby the distance of the membrane 74 to the round window membrane 88 is adjustable by rotation of the support frame 70 relative to the fixation element 90. The flange portion 92 may comprise a shape memory alloy, a shape memory resin or a bendable spring structure in order to fix the fixation element 90 within the round window opening. Alternatively, the flange portion 92 may comprise a titanium fixation plate fixed to the cochlea wall 22 by appropriate screws.

According to the above-mentioned article by S. Rosset et al., a deflection of more than 150 μm can be obtained for a chip having an opening having a diameter of 2 mm. However, the actuation voltage would be too high for a human implant. Nevertheless, according to Nakajima, H. H., et al. “Performance considerations of prosthetic actuators for round-window stimulation”. Hear. Res., 2009, a deflection of 100 nm would be enough to stimulate the round window and create a cochlear response equivalent to a normal sound stimulation of 100 dB SPL. The estimated maximum needed force to apply on the round window is about 300 μN. This force and displacement estimations represent a mechanical work of about 30 pJ. According to the above-mentioned article by S. Rosset et al, the maximum mechanical work of a 2 mm diameter membrane was about 100 nJ under 1400 V. As the needed mechanical work is several order of magnitude smaller, it could be obtained with a much lower voltage level (<100 V) which is compatible with an implantable power supply.

According to an alternative embodiment, the actuator 20 may be designed to float within the cochlear liquid (perilymph), with the membrane 70 directly touching the cochlear liquid, thereby directly vibrating the cochlear liquid. Such an embodiment is schematically indicated in FIG. 2. For such inner ear actuator directly mechanically stimulating the cochlea liquid, the necessary volume displacement is about 2.6 n/; such volume displacement is achievable with the actuators according to the present invention.

According to a variant, the polymer membrane actuator could be integrated within a cochlear implant electrode arrangement for achieving both electrical and acoustic stimulation of the cochlea. A schematic example is shown in FIG. 7, wherein a cochlear implant electrode assembly 100 is provided with a polymer membrane actuator 120, e.g. similar to the actuator type shown in FIG. 5, which is integrated within the cochlear implant electrode assembly 100 in manner that the membrane of the actuator 120 touches the cochlear liquids for acoustic stimulation of the cochlea 24. The individual electric stimulation contacts 102 of the electrode assembly 100 are arranged, as usual, spaced apart from each other along the length of the electrode assembly 100.

While various embodiments in accordance with the present invention have been shown and described, it is understood that the invention is not limited thereto, and is susceptible to numerous changes and modifications as known to those skilled in the art. Therefore, this invention is not limited to the details shown and described herein, and includes all such changes and modifications as encompassed by the scope of the appended claims.

1. An implantable actuator for stimulating a patient's hearing, comprising a support frame, a dielectric electroactive polymer membrane fixed and tensioned on the support frame, with the polymer membrane being provided with a first electrode on one side and a second electrode on another side, means for supplying electric signals to the electrodes, wherein the polymer membrane is for vibrating a cochlear liquid according to the electric signals supplied to the electrodes by direct contact or by contact with a round window membrane.

2. The actuator of claim 1, wherein the polymer membrane is made of polydimethylsiloxane.

3. The actuator of claim 1 wherein the support frame comprises silicon or glass.

4. The actuator of claim 1, wherein the electrodes are soft electrodes, each formed by an ion implanted surface layer of the polymer membrane.

5. The actuator of claim 4, wherein the ion implanted surface layer of the polymer membrane comprises Ti-ions.

6. The actuator of claim 1, wherein the support frame and the audio signal supplying means are covered with bio-compatible cover layer.

7. The actuator of claim 6, wherein the cover layer comprises one of PEEK, titanium, silicone and PMMA.

8. The actuator of claim 1, wherein the support frame comprises a silicon chip comprising an opening covered by the polymer membrane.

9. The actuator of claim 1, further comprising means for fixing the support frame at a round window in such a manner that the polymer membrane touches the round window membrane for vibrating the round window membrane according to the electric signals supplied to the electrodes.

10. The actuator of claim 9, wherein the fixing means comprises an auxiliary fixation element which is fixed within the round window opening in a cochlear wall.

11. The actuator of claim 10, wherein the support frame engages with the auxiliary fixation element in a manner that an axial position of the support frame relative to the fixation element is adjustable in order to the adjust a distance of the polymer membrane to the round window membrane.

12. The actuator of claim 11, wherein the support frame is to be screwed into the auxiliary fixation element, with the auxiliary fixation element comprising an internal thread engaging with an outer thread of the support frame.

13. The actuator of claim 12, wherein the auxiliary fixation element comprises an outer flange portion for abutting the cochlear wall and an inner cylindrical portion for extending into the round window opening, with the inner cylindrical portion being provided with the internal thread.

14. The actuator of claim 1, wherein the actuator is designed for floating within the cochlear liquid, with the polymer membrane touching the cochlear liquid.

15. The actuator of claim 1, wherein the actuator is integrated within a cochlear implant electrode.

16. An at least partially implantable hearing instrument comprising an actuator of claim 1, further comprising a microphone arrangement for capturing audio signals from ambient sound and an audio signal processing unit for processing the captured audio signals.

17. The hearing instrument of claim 16, wherein the microphone arrangement and the audio signal processing unit form part of an external unit to be worn at a patient's body, and wherein the external unit comprises means for transmitting the processed audio signals via a transcutaneous audio link to an implantable unit connected by wires to the actuator.

18. The hearing instrument of claim 17, wherein the external unit comprises a power source and means for transmitting power via a transcutaneous power link (60) to the implantable unit.

19. A method of providing hearing assistance to a patient, by using an implantable actuator comprising a support frame, a dielectric electroactive polymer membrane fixed and tensioned on the support frame, with the polymer membrane being provided with a first electrode on one side and a second electrode on another side, the method comprising:
supplying audio signals to the electrodes and vibrating, via the polymer membrane, a cochlea liquid according to the audio signals supplied to the electrodes by direct contact or by contact with the round window membrane, thereby stimulating a patient's hearing.
